(19) United States
(12) Patent Application Publication
(10) Pub. No.: US 2011/0245779 A1
(43) Pub. Date: Oct. 6, 2011

(54) Fluid Valve Systems

(76) Inventor: John Hiebert, Wayland, MA (US)

(21) Appl. No.: 13/161,564

(22) Filed: Jun. 16, 2011

Related U.S. Application Data
(63) Continuation-in-part of application No. 11/692,401, filed on Mar. 28, 2007, now Pat. No. 7,975,721.
(60) Provisional application No. 60/786,883, filed on Mar. 30, 2006.

(51) Int. Cl. A61M 5/168

(52) U.S. Cl. 604/186

(57) ABSTRACT

An apparatus includes a first valve having an inlet capable of being in fluid communication with a first fluid source and an outlet capable of being in fluid communication with the inlet. The first valve is capable of being in a first mode in which the inlet is in fluid communication with the outlet through a first flow path, and a second mode in which the inlet is in fluid communication with the outlet through a second flow path isolated from the first flow path.

### CROSS-REFERENCE TO RELATED APPLICATIONS

This application is a Continuation-in-Part of U.S. patent application Ser. No. 11/692,401, filed on Mar. 28, 2007, which claims benefit of U.S. Provisional Patent Application Ser. No. 60/786,883, filed on Mar. 30, 2006. Each patent and patent application cited herein is hereby incorporated by reference in its entirety.

### FIELD OF THE INVENTION

The invention relates to fluid valve systems, such as an apparatus for controlling the intravenous administration of fluid.

### BACKGROUND

In hospitals, it is common to administer fluid and medicaments to patients via the parenteral or intravenous (IV) route. The vast majority of hospitalized patients and all patients undergoing surgery have an IV catheter placed for delivering medicaments such as antibiotics or emergency resuscitation drugs, for inducing and maintaining anesthesia, and for the infusion of fluid volume and blood products.

Two broad functional modes define IV systems: continuous infusion and bolus dosing. The bolus mode is used for medications that are safe for rapid administration and intended for immediate bioavailability. Bolus mode is typically quickly accessible within any IV system, because patients routinely are given a variety of medication boluses in addition to one or more concurrent infusions. The frequency of bolus drug administration may range from zero to dozens per day. Occasionally, multiple boluses are delivered in rapid succession necessitating repeated switching between modes, as occurs in surgery and interventional procedures, in the intensive care unit (ICU), in the management of unstable patients, and in medical and surgical emergencies.

### SUMMARY

The invention relates to fluid valve systems, such as, for example, IV systems so as to increase safety and efficiency of IV administration of fluid and medicaments.

In one aspect, the invention features an apparatus including a first valve having an inlet capable of being in fluid communication with a first fluid source; and an outlet capable of being in fluid communication with the inlet, wherein the first valve is capable of being in a first mode in which the inlet is in fluid communication with the outlet through a first flow path, and a second mode in which the inlet is in fluid communication with the outlet through a second flow path isolated from the first flow path.

Embodiments may include one or more of the following features. The first valve further includes a port in fluid communication with the second flow path. The port is adapted to be in fluid communication with an injector. The first valve is associated with a base, and the injector is capable of pivoting relative to the base. The apparatus further includes an injector undetachably attached to the first valve. The apparatus further includes an injector engageable with the first valve, and an enclosure enclosing at least a portion of the injector. The first valve further includes a controller capable of controlling fluid flow through the first flow path. The first flow path is capable of being in fluid communication with a second fluid source. The first valve includes a body having at least a portion of the first flow path and at least a portion of the second flow path, and rotation of the body allows the first valve to be in a selected mode. The first valve includes a body having a first channel, a second channel, and a third channel, the first, second, and third channels capable of being isolated from each other, wherein, in the first mode, the first channel, the second channel, the inlet, the outlet, and the first flow path are in fluid communication, and in the second mode, the third channel, the inlet, and the outlet are in fluid communication. The first valve further includes a tactile indicator capable of denoting a selected mode of the first valve.

The apparatus can further include a second valve capable of being in fluid communication with the first valve, the second valve including a second inlet, a second outlet capable of being in fluid communication with the second inlet, and a port, wherein the second valve is capable of being in a third mode in which the second inlet is capable of being in fluid communication with the second outlet, and a fourth mode in which the second outlet is capable of being in fluid communication with the port. The second valve can be configured to engage with a fluid injector, and the injector and the port are capable of being in fluid communication. The second valve can be associated with a base, and the injector is capable of pivoting relative to the base. The first and second valves can be secured to a base.

In one aspect, the invention features an apparatus including a first valve having an inlet capable of being in fluid communication with a first fluid source; and an outlet capable of being in fluid communication with the inlet, wherein the first valve is capable of being in a first mode in which the inlet is in fluid communication with the outlet through a first flow path, and a second mode in which the inlet is in fluid communication with the outlet through a second flow path isolated from the first flow path. The apparatus further includes one or more subsequent valves (i.e., a second valve, a third valve, etc.) constructed the same as the first valve and in fluid communication therewith. The second valve and/or any subsequent valve can be configured to engage with a fluid injector so as to be capable of being in fluid communication therewith. The second valve and/or any subsequent valve can be configured to include an optional midstream conduit. The second valve and/or any subsequent valve can be associated with a base, and the injector is capable of pivoting relative to the base. The first and second valves can be secured to a base.

In another aspect, the invention features an apparatus, including a first valve having an inlet capable of being in fluid communication with a first fluid source; and an outlet capable of being in fluid communication with the inlet, wherein the first valve is capable of being in a first mode in which the inlet is in fluid communication with a first flow path, and a second mode in which the inlet is in fluid communication with the outlet through a second flow path isolated from the first flow path; and a second valve capable of being in fluid communication with the first valve, the second valve including a second inlet, a second outlet capable of being in fluid communication with the second inlet, and a port, wherein the second valve is capable of being in a third mode in which the second inlet is capable of being in fluid communication with the second outlet, and a fourth mode in which the second outlet is capable of being in fluid communication with the port, wherein the first flow path extends from the inlet of the first valve to a position downstream of the second outlet of the second valve when the first valve is in the second mode.

Embodiments may include one or more of the following features. The apparatus further may include one or more optional unidirectional valves along the first flow path. The first valve further includes a port in fluid communication with the second flow path. The port is adapted to be in fluid communication with an injector. The first valve is associated with a base, and the injector is capable of pivoting relative to the base. The first valve further includes a controller capable of controlling fluid flow through the first flow path. The first flow path is capable of being in fluid communication with a second fluid source. The first valve includes a body having at least a portion of the first flow path, at least a portion of the second flow path, and one or more unidirectional valves, and rotation of the body allows the first valve to be in a selected mode. The first valve further includes a tactile indicator capable of denoting a selected mode of the first valve.

In another aspect, the invention features a method, including in a first mode, flowing a fluid from an inlet of a first valve to an outlet of the first valve through a first flow path; and in a second mode, flowing the fluid from the inlet to the outlet through a second flow path isolated from the first flow path.

Embodiments may include one or more of the following features. Flowing the fluid from the inlet to the outlet through the second flow path includes flowing the fluid along the second flow path. The method further includes controlling the rate of flow through the first flow path. The method further includes introducing a second fluid to the first flow path. The method further includes rotating a body of the valve to select a mode. The method further includes drawing fluid from the inlet into the second flow path. The method further includes delivering fluid from the second flow path to the outlet. The method further includes tactilely detecting a selected mode of the valve. The method further includes flowing the fluid from the outlet to a second valve.

Embodiments may include one or more of the following features. Flowing the fluid from the inlet to the outlet through the second flow path includes flowing the fluid through one or more optional unidirectional valves along the second flow path. The method further includes controlling the rate of flow through the first flow path. The method further includes introducing a second fluid to the first flow path. The method further includes rotating a body of the valve to select a mode. The method further includes drawing fluid from the inlet into the second flow path. The method further includes delivering fluid from the second flow path to the outlet. The method further includes tactilely detecting a selected mode of the valve. The method further includes flowing the fluid from the outlet to a second valve.

In another aspect, the invention features a method, including in a first mode, flowing a fluid from an inlet of a first valve to a patient without passing the fluid through an outlet of the first valve, the fluid flowing through a first flow path; and in a second mode, flowing the fluid from the inlet to the outlet through a second flow path isolated from the first flow path.

Embodiments may include one or more of the following features. Flowing the fluid from the inlet to the outlet through the second flow path includes flowing the fluid along the second flow path. The method further includes controlling the rate of flow through the first flow path. The method further includes introducing a second fluid to the first flow path. The method further includes rotating a body of the valve to select a mode. The method further includes drawing fluid from the inlet into the second flow path. The method further includes delivering fluid from the second flow path to the outlet. The method further includes tactilely detecting a selected mode of the valve. The method further includes flowing the fluid from the outlet to a second valve. In the first mode, fluid flows from the inlet to a location downstream of fluid exiting the second valve. The method further includes flowing fluid through the first flow path.

Embodiments may include one or more of the following features. Flowing the fluid from the inlet to the outlet through the second flow path includes flowing the fluid through one or more optional unidirectional valves along the second flow path. The method further includes controlling the rate of flow through the first flow path. The method further includes introducing a second fluid to the first flow path. The method further includes rotating a body of the valve to select a mode. The method further includes drawing fluid from the inlet into the second flow path. The method further includes delivering fluid from the second flow path to the outlet. The method further includes tactilely detecting a selected mode of the valve. The method further includes flowing the fluid from the outlet to a second valve. In the first mode, fluid flows from the inlet to a location downstream of fluid exiting the second valve. The method further includes flowing fluid through the first flow path.

In another aspect, the invention may feature an apparatus for directing intravenous fluid flow to a subject, the apparatus including: a first valve sized and dimensioned for directing the flow of intravenous fluid including: an inlet capable of being in fluid communication with an intravenous fluid source; an outlet capable of being in fluid communication with the inlet; and a first port adapted to be in fluid communication with a syringe, wherein the first valve has an axis of rotation and is capable of being in a first mode in which the inlet is in fluid communication with the outlet through a first flow path, and a second mode in which the inlet is in fluid communication with the outlet through a second flow path isolated from the first flow path, the first port is in fluid communication with the second flow path, and the syringe, when coupled to the first port, provides a first valve handle for selecting between the first mode and second mode by rotation of the syringe about the axis of rotation of the first valve; and a second valve sized and dimensioned for directing the flow of intravenous fluid capable of being in fluid communication with the first valve, the second valve including: a second inlet; a second outlet capable of being in fluid communication with the second inlet; and a second port adapted to be in fluid communication with a second syringe, wherein the second valve has an axis of rotation and is capable of being in a third mode in which the second inlet is capable of being in fluid communication with the second outlet, and a fourth mode in which the second outlet is capable of being in fluid communication with the second port, and the second syringe, when coupled to the second port, provides a second valve handle for selecting between the third mode and the fourth mode by rotation of the second syringe about the axis of the rotation of the second valve, wherein the first flow path extends from the inlet of the first valve to a position downstream of the second outlet of the second valve when the first valve is in the second mode.

Embodiments may include one or more of the following advantages.

The valve systems can replace and/or supersede the functions of a four-way stopcock.

The valve systems can serve conveniently the two main functions of an IV infusion system, those being: (1) baseline continuous flow from a main IV fluid and any auxiliary IV fluid; and (2) rapid bolus injection and flushing, for example, of medicament. In some embodiments, the systems achieve the above by incorporating two distinct types of valve: an upstream valve containing two or more mutually exclusive fluid streams, which rotates to one position for flush syringe filling and emptying and to another position for constant infusion flow from the main IV fluid reservoir and any auxiliary IV fluid reservoirs; and a downstream valve which rotates to either of two positions to achieve exclusive bolus injection (e.g., of medicament) or infusion flow from one or more IV infusion bags.

The valve systems can promote safety of patients, for example, in the following ways: (1) by providing one-handed operation, leaving the other hand of the clinician free to perform other tasks; (2) by allowing syringes filled with medicament to remain attached to the medicament injection valves without risking inadvertent injection and without needing to use single valves for multiple purposes, such as flushing or connection of auxiliary infusions; (3) by restricting medicament injection valves to only two rotational settings, neither of which allows medicament to come into contact with the upstream fluid pathway, thereby precluding contamination or dilution of medicament; and/or (4) avoiding inadvertent fluid overload as a result of improved flush technique.

The valve systems can promote safety of clinicians by simplifying the filling of flush syringes, thereby reducing the tendency of a clinician to choose a needle-based method of flush syringe filling.

The valve systems can promote efficiency of clinicians, for example, in the following ways: (1) by providing one-handed operation for all valve functions, including medicament injection, flush syringe filling, and flush syringe emptying; (2) by reducing the number of steps used to inject and flush medicament and by making those steps easier to perform; (3) by permitting full-bore flush syringe filling and emptying without needing to make any prior adjustment to a roller clamp; (4) by permitting flush syringe filling from the main IV fluid without needing to clamp any auxiliary IV fluid line beforehand; (5) by establishing a uniform and predictable location in the IV setup where auxiliary infusion(s) are intended to be connected; and (6) by allowing valve position to be determined easily by gross visual inspection of the angle of the attached syringe as opposed to looking at the lever position of a conventional stopcock.

The valve systems can be made by press fit assembly of injection molded plastic components to provide a sterile, disposable system. In order to facilitate one-handed operation, the valves may be attached to a base of dimensions suitable to allow rotation of the valves by moving the attached syringes while the base serves as an anchor. The valves can be attachable in one embodiment by Luer lock connectors so that multiple medicament injection valves may be placed in series downstream from the flush valve.

### BRIEF DESCRIPTION OF THE DRAWINGS

These and other features of the present embodiments will be understood better by reading the following detailed description, taken together with the figures herein described. The accompanying drawings are not intended to be drawn to scale. In the drawings, each identical or nearly identical component that is illustrated in various figures is represented by a like numeral. For purposes of clarity, not every component may be labeled in every drawing.

FIG. 1 illustrates a front elevation view of an embodiment of a valve system showing one flush valve and three injection valves, and showing a second injection valve in the injection mode. Elements are not shown in the same scale. Three auxiliary infusions are shown with their tubing leading toward a midstream conduit (not shown) of the flush valve.

FIG. 2 illustrates a top view of the valve system shown in FIG. 1.

FIG. 3A illustrates detail of a front elevation view of the flush valve in drip mode.

FIG. 3B illustrates detail of a top view of the flush valve in drip mode.

FIG. 3C illustrates the valve system of FIG. 3A further including one or more optional unidirectional valves.

FIG. 3D illustrates the valve system of FIG. 3B further including one or more optional unidirectional valves.

FIG. 4 illustrates an exploded perspective view of the flush valve in drip mode.

FIG. 5 illustrates a perspective view of the flush valve in drip mode.

FIG. 6A illustrates a front view of a flush cylinder, showings its one port and associated luer connector, and also showing the cylinder's channels.

FIG. 6B illustrates a top view of the flush cylinder of FIG. 6A.

FIG. 6C illustrates a front perspective view of FIG. 6A showing the channel openings on the surface of the flush cylinder.

FIG. 6D illustrates a top perspective view of the flush cylinder of FIG. 6A.

FIG. 6E illustrates a left side view of the flush cylinder of FIG. 6A.

FIG. 7A illustrates a side view of the flush valve with flush syringe attached, in drip-flow mode.

FIG. 7B illustrates an exploded left side view of the flush valve with the flush cylinder removed therefrom and positioned above the end cap.

FIG. 7C illustrates an exploded perspective view of the flush valve in drip-flow mode.

FIG. 8A illustrates a side view of the flush valve with flush syringe attached, in flush-fill mode.

FIG. 8B illustrates en exploded left side view of the flush valve with the flush cylinder removed therefrom and positioned above the end cap.

FIG. 9A illustrates a side view of the flush valve with flush syringe attached, in flush-purge mode.

FIG. 9B illustrates an exploded left side view of the flush valve with the flush cylinder removed therefrom and positioned above the end cap.

FIG. 10 illustrates an exploded perspective view of the flush valve in flush mode.

FIG. 11 illustrates an exploded perspective view of the injection valve in drip mode.

FIG. 12 illustrates detail of a top view of the injection valve in drip mode.

FIG. 13 illustrates an exploded perspective view of the injection valve in injection mode.

FIG. 14 illustrates a top view of an embodiment of a valve.

### DETAILED DESCRIPTION

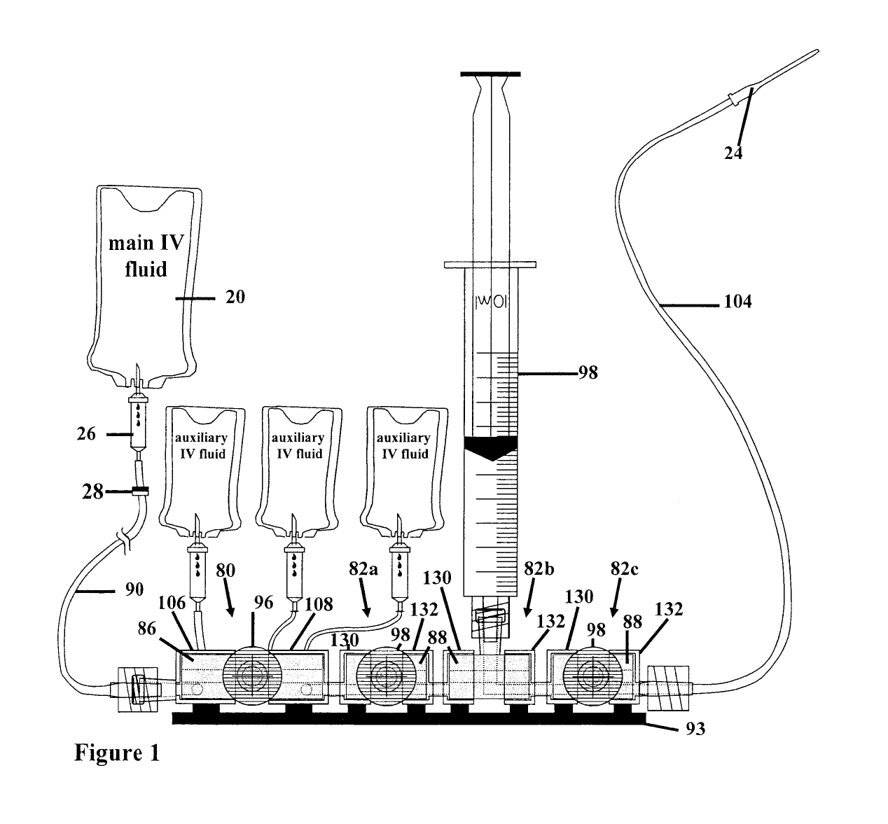

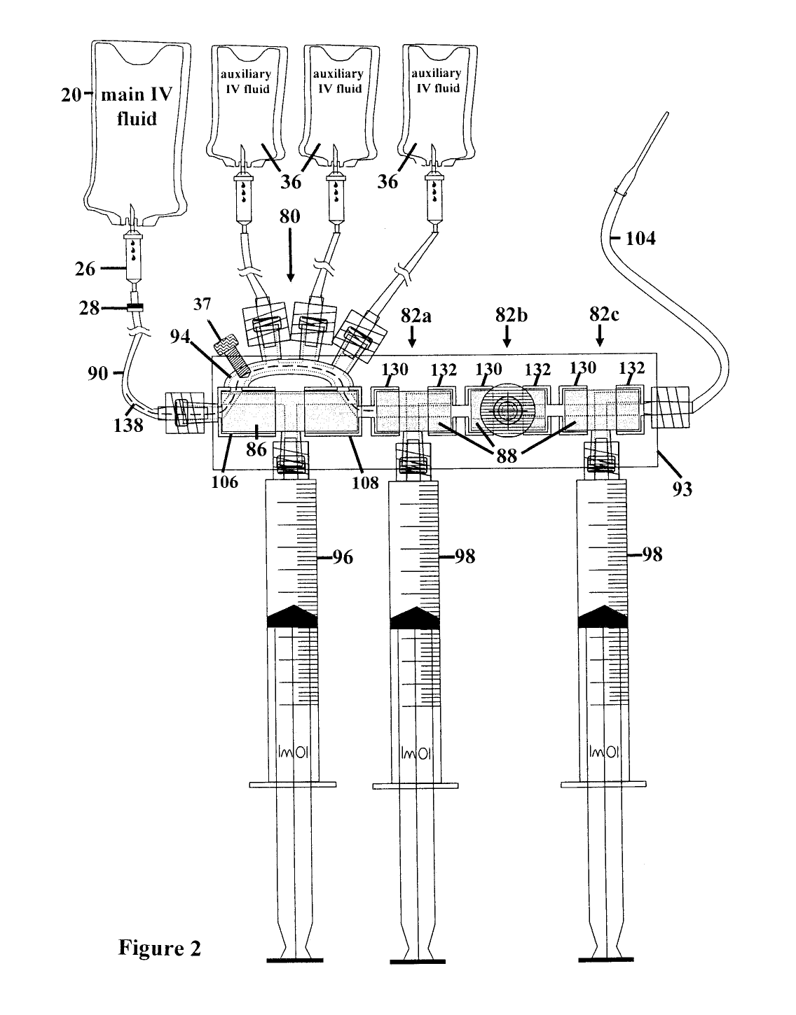

FIGS. 1 and 2 depict an apparatus for regulating fluid flow in an IV administration system—FIG. 1 from the front, and FIG. 2 from the top. Fluid is received from a main IV fluid reservoir 20 via an upstream conduit 90, and/or from one or more auxiliary IV fluid reservoirs 36. Fluid is delivered subsequently to a downstream conduit 104 and onward to a patient (not shown). The apparatus includes two types of cylindrical valve: flush valve 80 and injection valve 82. Three injection valves 82a, 82b, and 82c are shown in FIGS. 1 and 2 arranged in series downstream of the flush valve. Each valve is actuated by movement of a syringe, such as syringes 96 and 98. Syringe 96 is attached to a flush cylinder 86, and syringe 98 is attached to an injection cylinder 88, such that the longitudinal axis of each syringe is perpendicular to the longitudinal axis of its associated cylinder. Any syringe shown in FIGS. 1 and 2 has the exclusive function of a flush syringe 96 or a medicament injection syringe 98, by virtue of the cylinder to which it is attached.

End caps are fluid-tight housings that fit snugly around the ends of the cylinders. The flush valve 80 has an upstream end cap 106 and a downstream end cap 108 positioned on the ends of flush cylinder 86. Injection valves have upstream end caps 130 and downstream end caps 132 positioned on the ends of injection cylinders 88. By moving the syringe in an arc, the clinician is able to rotate the attached cylinder, such as cylinder 86 and 88, about its longitudinal axis, causing channels within each cylinder to move relative to openings in end caps, and thereby actuate the valve to achieve a desired functional mode. Thus, fluid communication is allowed between an end cap opening and a cylinder channel only if they are in alignment. The syringes depicted in FIGS. 1 and 2 are not all positioned in the same orientation, illustrating their rotational mobility, and illustrating that each syringe is the actuator of rotation of its associated valve.

In some embodiments, valve cylinders are molded of a first plastic material, and end caps are molded of second plastic material to be assembled by press fit assembly, with a fluid tight seal present between the cylinder ends and sides and the end cap walls. As shown, the valve end caps are affixed to a wide base 93 to allow single-handed actuation of the valves using the syringes as lever arms. As shown, all four valves are affixed to a single base, as is depicted in FIGS. 1 and 2. Any valve or valve section depicted in other figures may be attached to a base or other support structure.

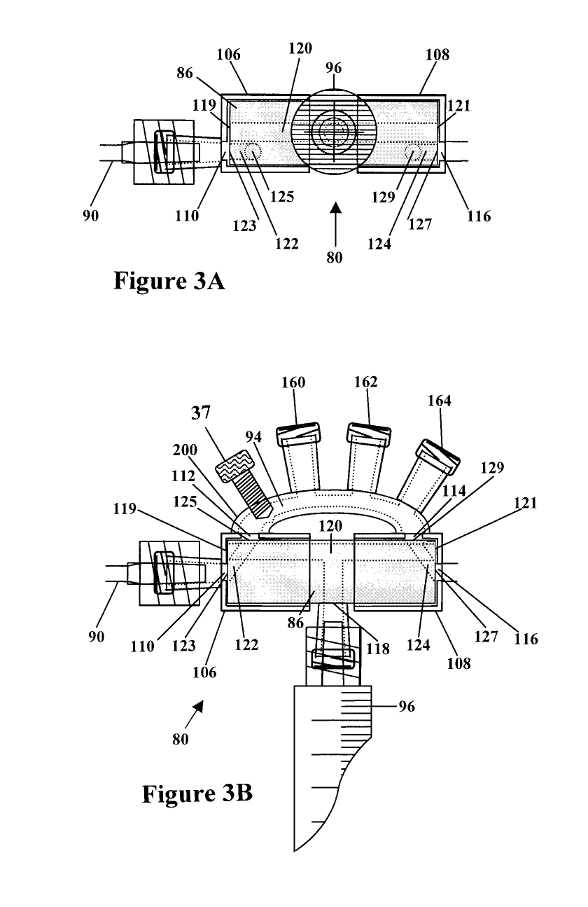

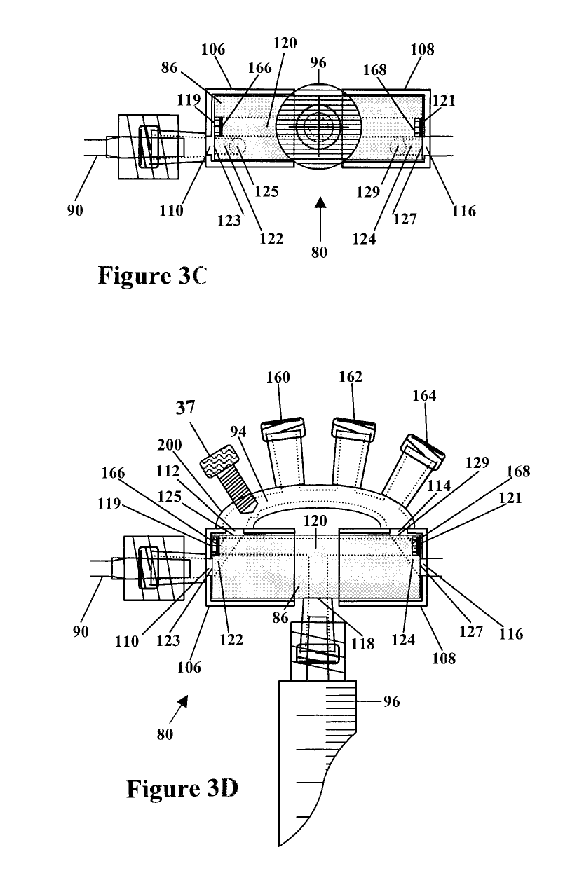

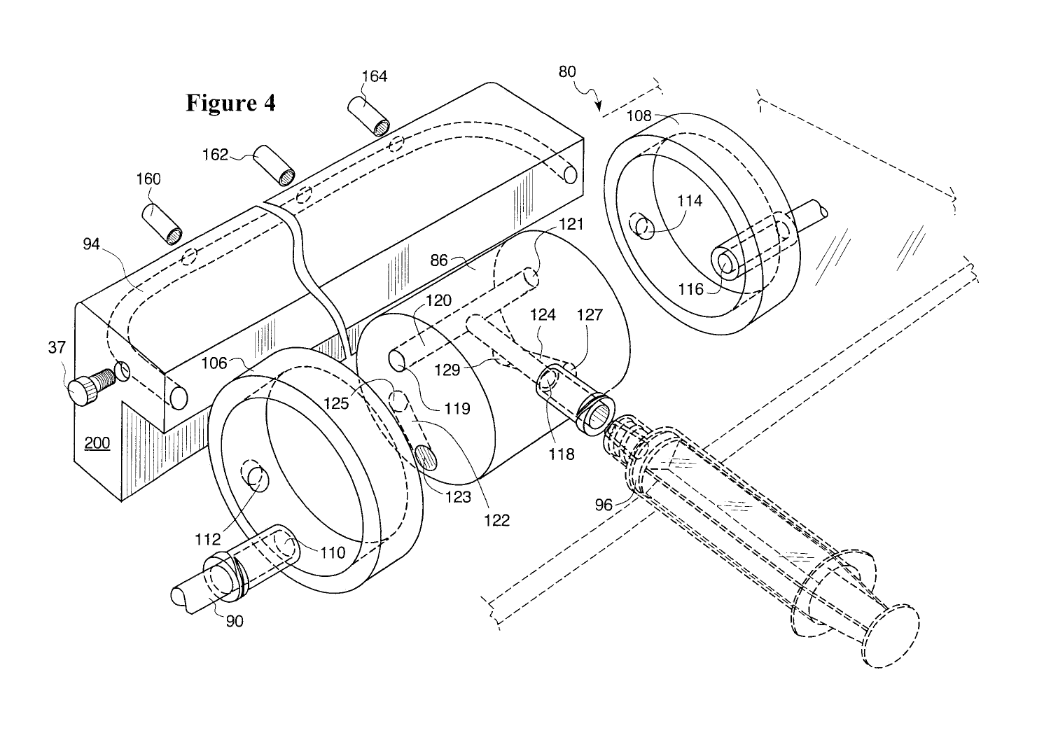

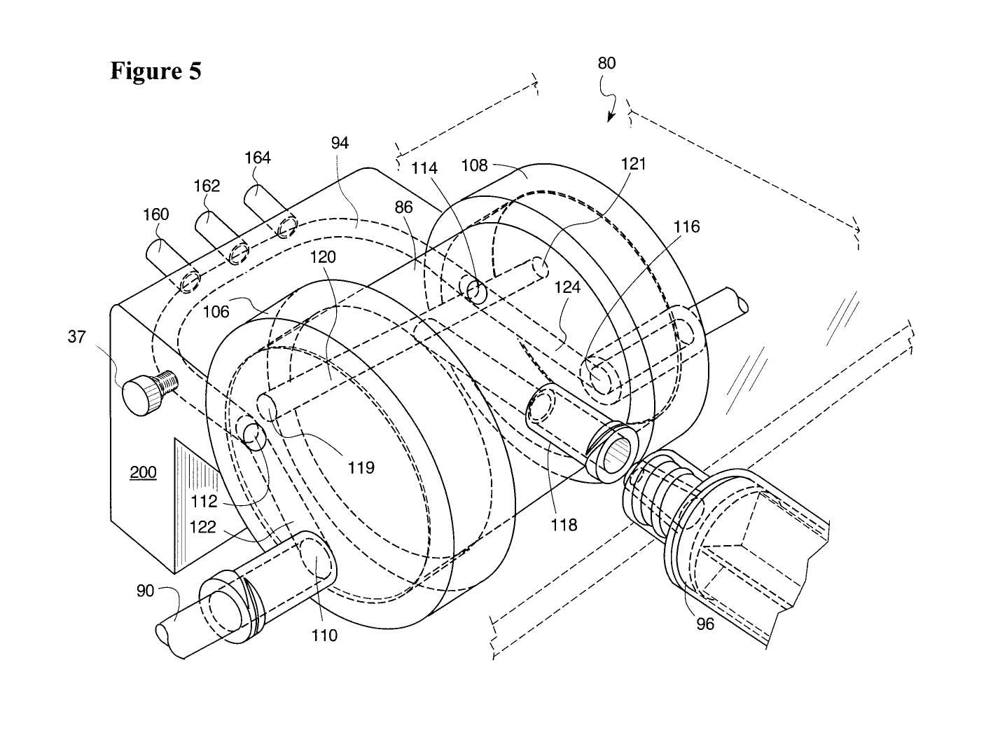

FIGS. 3A and 3B depict the flush valve 80 from the front and top, respectively, as in FIGS. 1 and 2, but in greater detail. FIGS. 4 and 5 show exploded and unexploded perspective views, respectively, of the same flush valve 80. The flush valve 80 includes an upstream end cap 106, a flush cylinder 86, and a downstream end cap 108. The upstream end cap has a first opening 110 and a second opening 112, and the downstream end cap has a first opening 114 and a second opening 116. The flush cylinder has a single port 118 on its side, with an attached female Luer connector, and is in fluid communication with a flush channel 120, which extends bi-directionally to both ends of the flush cylinder, from an upstream opening 119 to a downstream opening 121. Any syringe attached to flush cylinder port 118 is referred to as a flush syringe 96 and is in fluid communication with flush channel 120. Flush cylinder 86 also has two other channels, called an upstream drip channel 122 and a downstream drip channel 124. Upstream drip channel 122 extends from an opening 123 on the upstream end of flush cylinder 86 to an opening 125 on the side of the flush cylinder 86, and downstream drip channel 124 extends from an opening 129 on the side of flush cylinder 86 to an opening 127 on the downstream end of the flush cylinder 86. Flush channel 120 is not in fluid communication with upstream drip channel 122 or downstream drip channel 124, regardless of the selected mode of the flush valve.

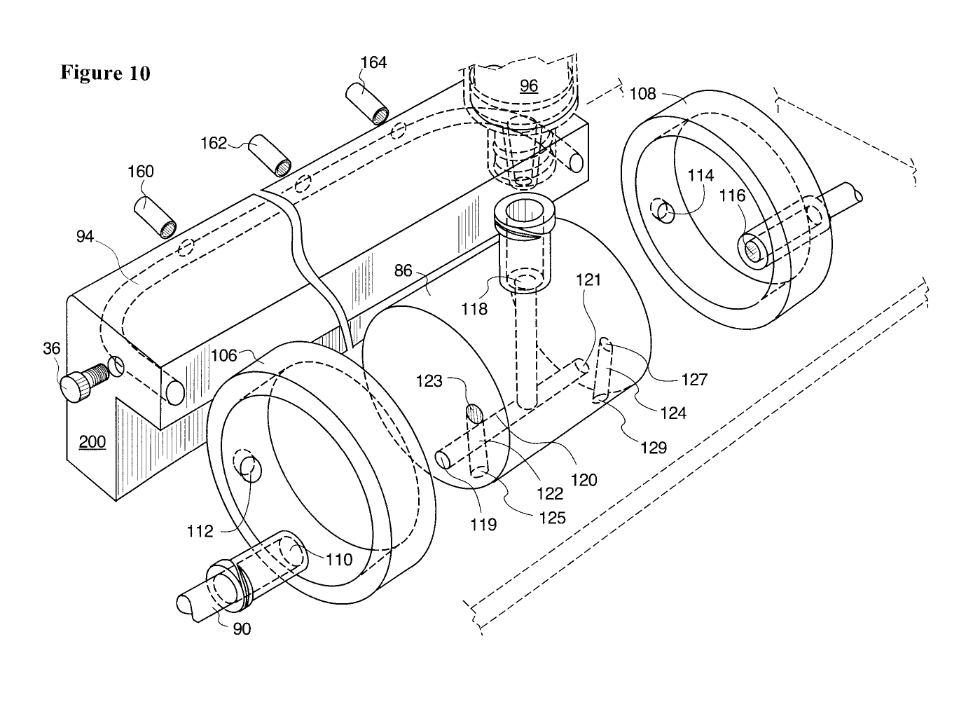

In one embodiment, flush valve 80 has two functional modes depending on the rotational position of flush cylinder 86: a drip mode and a flush mode. FIGS. 1, 2, 3A, 3B, 4, and 5 depict the flush valve in drip mode, with FIG. 10 showing an exploded view. In drip mode, flush syringe 96 resides in a horizontal position, parallel with the plane of base 93. This causes openings 123 and 125 of upstream drip channel 122 to align with upstream end cap openings 110 and 112, respectively, and openings 127 and 129 of downstream drip channel 124 to align with openings 116 and 114, respectively. Drip mode establishes a fluid path 138, shown in FIG. 4, in which fluid travels from upstream conduit 90, through first opening 110, into upstream drip channel 122, through second opening 112, into midstream conduit 94, through opening 168, into downstream drip channel 124, through opening 127, and onward to the patient.

Drip mode can be used whenever continuous (drip) infusion is needed, whether deriving from the main IV fluid reservoir 20, auxiliary infusion 36, or both. Drip mode permits standard modifications to the continuous fluid stream. One of these modifications is regulation of flow rate using a threaded screw valve 37 that variably regulates flow rate through the midstream conduit depending on the rotational position of the threaded screw. Another modification is the introduction of auxiliary IV fluid from reservoirs 36 by way of ports 160, 162, and 164. In some embodiments, ports 160, 162, and 164 contain integral gravity-fed one-way check valves (similar to those manufactured by Quest Medical, Inc., Allen, Tex.) to prevent retrograde flow, allowing only unidirectional flow from auxiliary IV fluid reservoirs 36 into midstream conduit 94. Screw valve 37 can eliminate the need for flow regulation elsewhere in the system, hence the absence of a roller clamp from upstream conduit 90 in FIGS. 1 and 2. The purpose of locating a flow regulator within the midstream conduit is to isolate its function and thereby provide flush syringe 96 with unimpeded, full-bore access to the main IV reservoir while the valve is in flush mode.

The flush mode of flush valve 80 is shown in FIG. 10 as an exploded perspective view. Flush mode is used to achieve rapid flushing of downstream conduit 104 using flush syringe 96. As shown, flush mode is activated when flush syringe 96 is positioned perpendicular to base 93, aligning flush channel opening 119 with upstream end cap opening 110 and aligning flush channel opening 121 with downstream end cap opening 116, simultaneously causing midstream conduit 94 to become unaligned from upstream drip channel 122 and downstream drip channel 124. Flush mode puts flush syringe 96 in fluid communication with upstream conduit 90 and with downstream conduit 104. Flush syringe 96 is filled by withdrawing the syringe plunger, thereby aspirating fluid from main IV fluid reservoir 20, and is emptied by depressing the syringe plunger, thereby forcing the syringe contents into downstream conduit 104.

In another embodiment, flush valve 80 may have three functional modes depending on the rotational position of flush cylinder 86: drip-flow mode, flush-fill mode, and flush-purge mode.

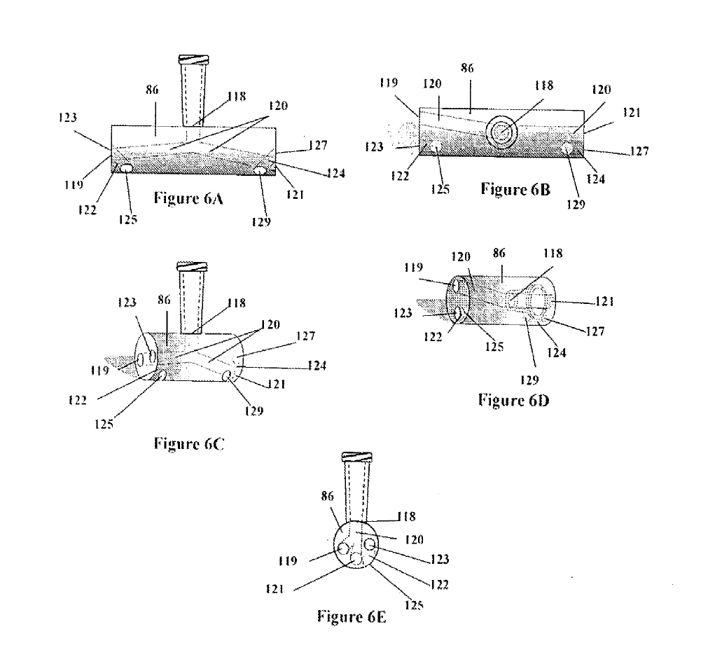

Referring to FIGS. 6A-6E, the flush valve 80 comprises an upstream end cap 106, flush cylinder 86, and a downstream end cap 108. The upstream end cap has first opening 110 and second opening 112, and the downstream end cap has first opening 114 and second opening 116. Each first opening may have a female luer connector and each second opening may have a male luer connector attached and in fluid communication therewith. The flush cylinder has a single port 118 on its side, with an attached female luer connector, and is in fluid communication with flush channel 120. The flush channel 120 extends to upstream opening 119 and downstream opening 121 in the flush cylinder. Because flush channel 120 is not parallel with the longitudinal axis of the flush cylinder, the two openings 119 and 121 of the flush channel are non-overlapping as seen in the left side view of FIG. 6E. This design enables exclusive flush syringe filling and exclusive flush syringe purging, as described below. Any syringe attached to flush cylinder port 118 is referred to as flush syringe 96. Flush syringe 96 is in fluid communication with flush channel 120, which extends bi-directionally to both ends of flush cylinder 86. Flush cylinder 86 also has two other channels, called upstream drip channel 122 and downstream drip channel 124, each of which extends from one end of flush cylinder 86 to the side of flush cylinder 86. Thus, upstream drip channel 122 extends from opening 123 seen in FIG. 6C on the upstream end of the flush cylinder 86 to opening 125 on the side of the flush cylinder, and downstream drip channel 124 extends from opening 129 on the side of flush cylinder 86 to opening 127 on the downstream end of flush cylinder 86.

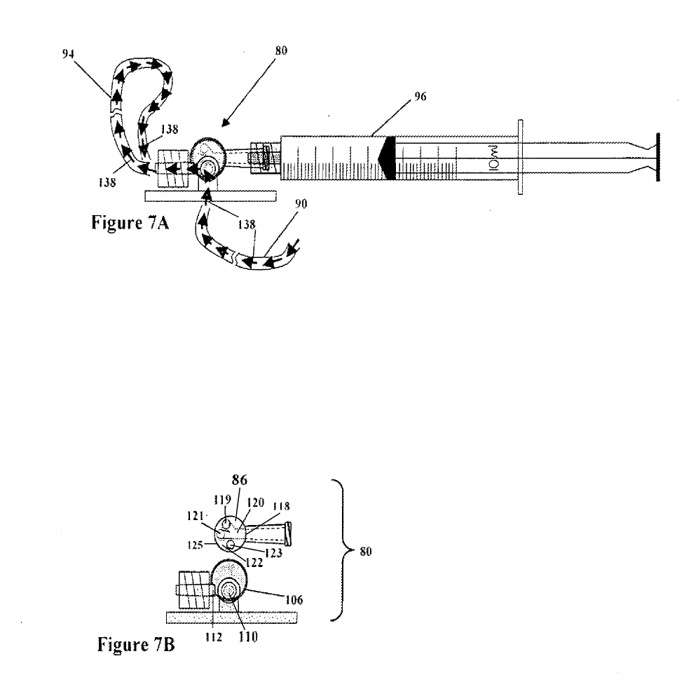

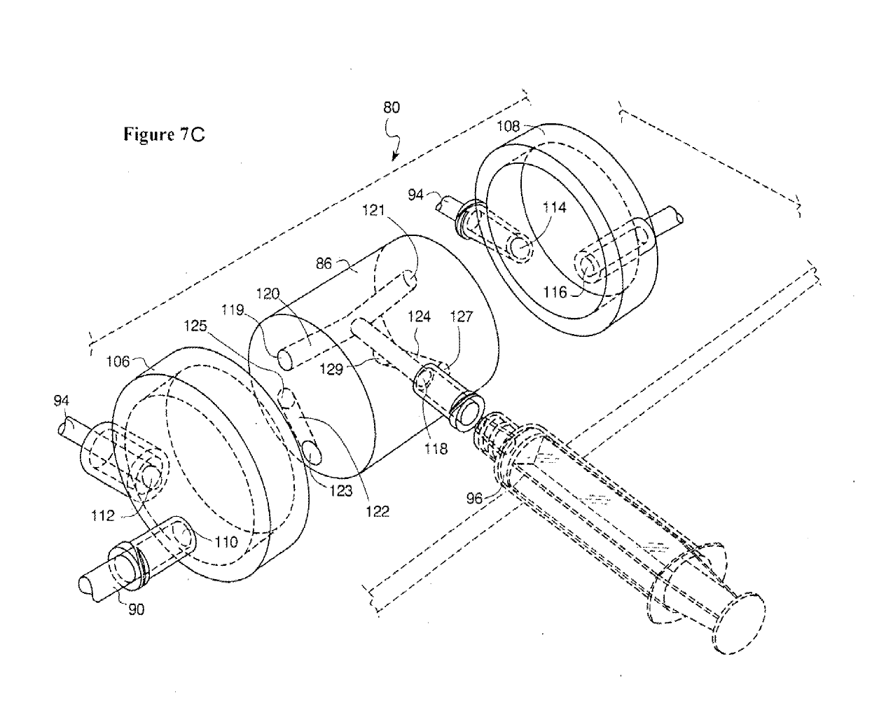

In drip-flow mode, flush syringe 96 resides in the horizontal position, as seen in FIGS. 7A-7B. The horizontal position orients the flush valve 80 so as to cause openings 123 and 125 of upstream drip channel 122 to come into register with openings 110 and 112, respectively, thereby creating fluid path 138, as seen in FIG. 2, in which fluid is conducted from upstream conduit 90 seen in FIG. 3B into first opening 110, into the upstream drip channel 122, out second opening 112, and into midstream conduit 94. The drip-flow mode also causes downstream drip channel openings 127 and 129 to come into register with second opening 116 and first opening 114, respectively, thereby completing fluid path 138, as seen in FIG. 2, in which fluid is conducted from midstream conduit 94, through downstream drip channel 124, and out of the flush cylinder via second opening 116. The function of the midstream conduit is to isolate the functions performed therein while the flush valve is not in drip-flow mode. In other words, flow rate regulation and auxiliary IV fluid addition are both excluded from the flow path when flush valve 80 is operating in either the flush-fill or flush-purge modes. In drip-flow mode, the fluid stream flows through the midstream conduit 94 where it may be subject to flow regulation, for example, by a roller clamp (not shown), and where it may merge with other fluid flow from auxiliary IV fluid reservoirs 36. In the drip-flow mode, no flow is permitted into or out of the flush syringe 96, because neither of the flush channel openings 119 and 121 seen in FIG. 6A are in register with any end cap opening.

FIG. 7C illustrates an exploded perspective view of flush valve 80 in its drip-flow mode with the flush cylinder disposed within the end caps and flush syringe 96 attached to flush cylinder port 118. In this position, the flush channel is not aligned with any openings so that its ends 119 and 121 face the upstream end cap 106 and downstream end cap 108, respectively, in a fluid-tight relationship while upstream drip channel 122 is aligned with first opening 110 and second opening 112, allowing fluid to pass from upstream conduit 90 to midstream conduit 94. In the same way, downstream drip channel 124 is aligned with the other end of midstream conduit 94 at side opening 129 to allow fluid to pass therethrough through downstream drip channel opening 127 into second opening 116 of the flush valve downstream end cap 108 and onward to the patient.

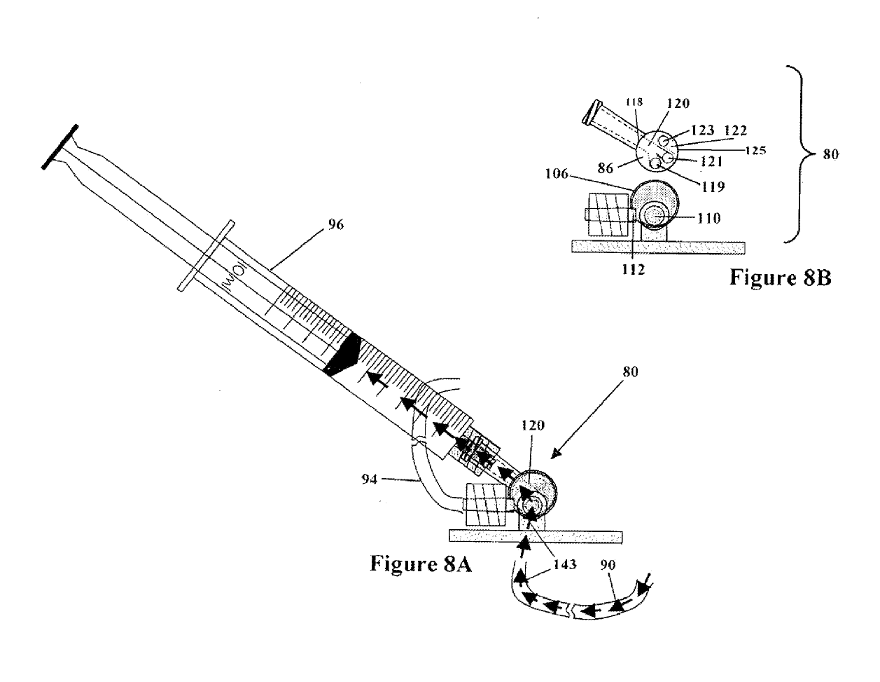

FIGS. 8A-8B show the flush-fill mode of flush valve 80. To bring about flush-fill mode, the flush syringe 96 is rotated to a position approximately 45 degrees past vertical, away from the user, allowing for filling of flush syringe 96 from the main IV fluid reservoir 20 by withdrawing the syringe plunger. Thus, the flush-fill mode creates fluid path 143, which permits flow exclusively from the main IV fluid reservoir 20, through the first opening 110 of the upstream end cap 106, into the upstream opening 119 of the flush channel 120, then into flush channel 120, through flush cylinder port 118, and into flush syringe 96. FIG. 8B shows an exploded, left-side view of the flush cylinder 86 positioned for flush-fill mode relative to upstream end cap 106.

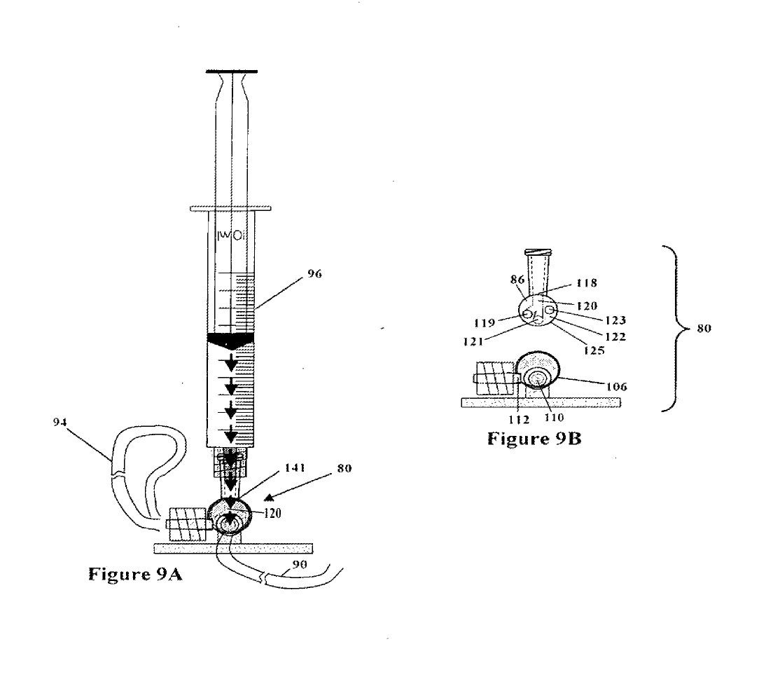

FIGS. 9A-9B show the flush-purge mode of flush valve 80. To bring about flush-purge mode, the flush syringe 96 is rotated to a vertical position, allowing for purging of flush syringe 96 toward the patient by depressing the syringe plunger. Thus, the flush-purge mode creates fluid path 141, which permits flow exclusively from the flush syringe 96, through flush cylinder port 118, into flush channel 120, through the downstream opening 121, through the second opening 116 in the downstream end cap 108, and onward to the patient. FIG. 9B shows an exploded, left-side view of the flush cylinder 86, positioned for flush-purge mode relative to upstream end cap 106.

There is no fluid communication between the midstream conduit 94 and flush channel 120 in any mode of operation. Thus, in both flush-fill and flush-purge modes of operation, the isolation of midstream conduit functions, such as, for example, flow regulation by a roller clamp and/or the introduction of auxiliary infusions, from the fluid path connected to the syringe relieves the clinician of the need to open such a roller clamp and/or to clamp any auxiliary IV fluid infusions prior to flush syringe filling or purging.

In an alternative embodiment, pathways 138 and 140 through the flush valve 80 need not be angled as shown in FIG. 8A; they can be straight. The reason they are angled in one embodiment is to allow co-linearity of the several valves. However, the valves need not be co-linear for the main functions of the invention to be achieved. Moreover, the various valves need not be connected by luer connectors, but can be connected by tubing or formed as a single unit.

The function of flush syringe 96 may be facilitated by the presence inside flush channel 120, in one embodiment, of one or more optional one-way check valves, though such a check valve is not required for the invention to function properly. In one particular embodiment, two pressure-activated, one-way check valves (similar to those manufactured by Quest Medical, Inc., Allen, Tex.) are included. As in FIGS. 3C and 3D, an upstream check valve 166 may be located on the end closest to upstream opening 119, and a downstream check valve 168 may be located on the end closest to downstream opening 121. The function of upstream check valve 166 is to prevent fluid in the flush channel from entering upstream conduit 90. The function of downstream check valve 168 is to prevent fluid in downstream conduit 104 from entering the flush channel. Pressure-activated check valves 166 and 168 require a sufficiently high pressure differential across the valves to open, such that a gravity-fed infusion would not cause the valves to open. Therefore, with flush valve 80 in flush mode, but with no force applied to the plunger of flush syringe 96, no flow would occur from the upstream fluid reservoir through the flush valve. Only with force applied upward or downward on the plunger of flush syringe 96 would fluid flow through openings 119 or 121, respectively. Thus, fluid can be aspirated from the upstream conduit 90 by withdrawing the plunger of flush syringe 96, but not from downstream conduit 104 because of downstream check valve 168. Similarly, fluid is forced into downstream conduit 104 by depressing the plunger of flush syringe 96, but not into upstream conduit 90 because of the presence of check valve 166. Thus, flush mode allows syringe 96 to act as a pump by withdrawing and depressing its plunger, causing fluid to move unidirectionally through flush channel 120. In neither drip mode nor flush mode does flush syringe 96 communicate with midstream conduit 94.

The midstream conduit 94 allows an infusion fluid stream to be mutually exclusive from a flushing stream, as shown, by providing the infusion fluid stream in parallel configuration with the flushing stream. Having two or more mutually exclusive fluid streams within one valve allows modification of one stream without affecting another. This is important, for example, when IV requirements dictate certain modifications to the fluid stream which are undesirable when the IV system is called upon to function in a different mode. For example, common IV fluid modifications include regulation of flow rate and the introduction of auxiliary infusions; however, these modifications can hinder efficient flushing of the line. Flush valve 80 facilitates these functions by sequestering those modifications in a separate fluid channel. Moreover, flush valve 80 simplifies the process by allowing rapid alternation between mutually exclusive fluid streams with a single motion, using the syringe as an actuator handle.

As shown, drip mode allows regulation of flow rate using screw valve 37 and the introduction of auxiliary IV fluid by way of ports 160, 162, and 164, but keeps flush channel 122 unaligned with any openings, because its openings 119 and 121 face the upstream end cap 106 and downstream end cap 108, respectively, in a fluid-tight relationship. Conversely, flush mode aligns flush channel openings 119 and 121 with end cap openings 110 and 116 to permit flush syringe filling and emptying, but keeps midstream conduit 94 unaligned with any openings.

As shown, the midstream conduit 94 is a fluid passageway formed inside a solid structure of molded plastic 200; however, in other embodiments, the midstream conduit takes a different form and still achieves the same function. For example, midstream conduit 94 could be formed of flexible plastic tubing extending from upstream end cap opening 112 to downstream end cap opening 114. Such tubing might be fitted with standard IV administration features such as a roller clamp, Y-sites, and/or stopcocks.

Similarly, in other embodiments, the flow controller in the midstream conduit takes a different form from the threaded screw valve 37. For example, a mechanism similar to a roller clamp might be employed. Another approach to flow rate control would be to close completely screw valve 37, so as to prohibit flow from upstream conduit 90 into midstream conduit 94. Then, an auxiliary IV fluid reservoir in fluid communication with the midstream conduit via ports 160, 162, or 164, and perhaps driven by a mechanical pump, could serve as the primary infusion. As a result, the main IV fluid reservoir 20 would function only as a flush reservoir, accessible only by flush syringe 96, and would not function as an infusion reservoir. An analog controller such as screw valve 37 need not be used, favoring instead the precision of electronically controlled infusion, but still retaining the advantages of an easy flushing system.

In other embodiments, the midstream conduit is not external to the flush cylinder. Rather, the midstream conduit can be integral to the flush cylinder, formed as a channel through the cylinder. Such a midstream conduit would be subjected to flow regulation and auxiliary fluid introduction just as described above, but those features would rotate along with the cylinder as the valve is actuated. Just as described above, the openings at the ends of the midstream conduit can come into alignment with end cap openings only in a specific rotational position of the flush cylinder.

Referring now to one of the injection valves 82 wherein the others, if used, are identical, three are shown in FIGS. 1 and 2, labeled 82a, 82b, and 82c. Each consists of a cylindrical valve element 88 which rotates within an upstream end cap 130 and a downstream end cap 132. In FIGS. 1 and 2, one of the three injection valves, 82b, is shown in injection mode; that is, with cylinder 88 positioned rotationally such that the attached syringe 98 is perpendicular to base 93. Injection valves 82a and 82c are shown in drip mode; that is, with their cylinders 88 positioned rotationally such that their attached syringes 98 are parallel to base 93.

In another embodiment, one or more injection valves 82 may be constructed similarly or identically to flush valve 80. In such an embodiment, the second valve and/or any subsequent valve may comprise the same type of valve as the first valve (i.e., flush valve 80). In one particular embodiment, the second valve and/or any subsequent valve, such valve(s) arranged similarly or identically to the first valve, may include an optional mid-stream conduit 94 in much the same way as with the first valve (i.e., flush valve 80), though such additional conduit(s) are not essential to functionality. The second valve and/or any subsequent valve may be arranged so as to include no mid-stream conduit 94. There may be more than one, more than two, more than three, more than four, or more than five such flush valves 80 arranged together, with one or more such valves capable of contributing to a flushing function and one or more such valves capable of contributing to an injection function, in much the same manner as injection valves 82. The one or more valve(s) constructed similarly or identically to flush valve 80 may be interspersed along the desired fluid path and need not be juxtaposed with one another. Such one or more flush valves may incorporate either of the two-functional-mode system or the three-functional-mode system previously discussed herein.

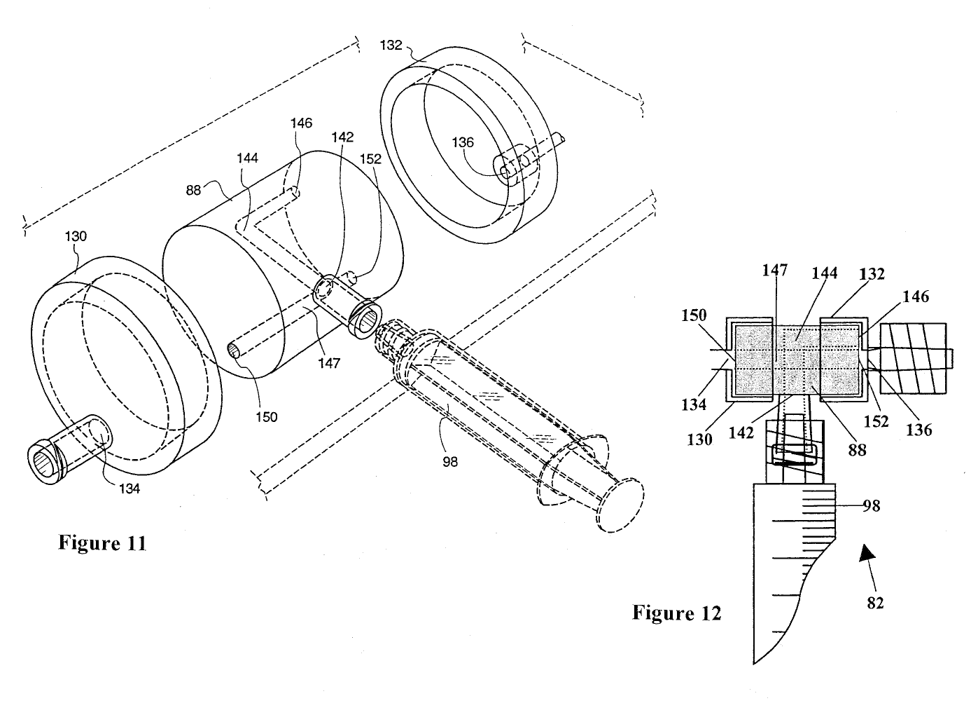

FIGS. 11 and 12 show injection valve 82 in drip mode, with FIG. 11 being an exploded perspective view, and FIG. 12 being a top view. Each end cap has an opening through which fluid may move only if aligned with an opening in the injection cylinder 88. Upstream end cap 130 has an opening 134, and downstream end cap 132 has an opening 136. Injection cylinder 88 has a port 142 on its side, with an attached female Luer connector, and fluidly communicates with an injection channel 144. Any syringe attached to port 142 is called an injection syringe 98. The injection syringe is in fluid communication with injection channel 144, which extends unidirectionally to an opening 146 at the downstream end of injection cylinder 88. Injection cylinder 88 has one other channel, called drip channel 147 which is a linear conduit, parallel with the longitudinal axis of the injection cylinder, extending from an opening 150 at the upstream end of injection cylinder 88 to an opening 152 at the downstream end of injection cylinder 88.

The injection valve has two functional modes, drip mode and injection mode, depending on the rotational position of the injection cylinder. In drip mode, the injection syringe is positioned parallel to the plane of base 93, as seen with valves 82a and 82c in FIGS. 1 and 2. This rotational position causes openings 150 and 152 of drip channel 147 to align with end cap openings 134 and 136 respectively. Drip mode allows fluid to be conducted into opening 134, through drip channel 147, out opening 136, and into the next injection valve, if one is present, and then into downstream conduit 104. Drip mode does not allow fluid communication between injection syringe 98 and the patient, because opening 146 of injection channel 144 is not aligned with any end cap opening.

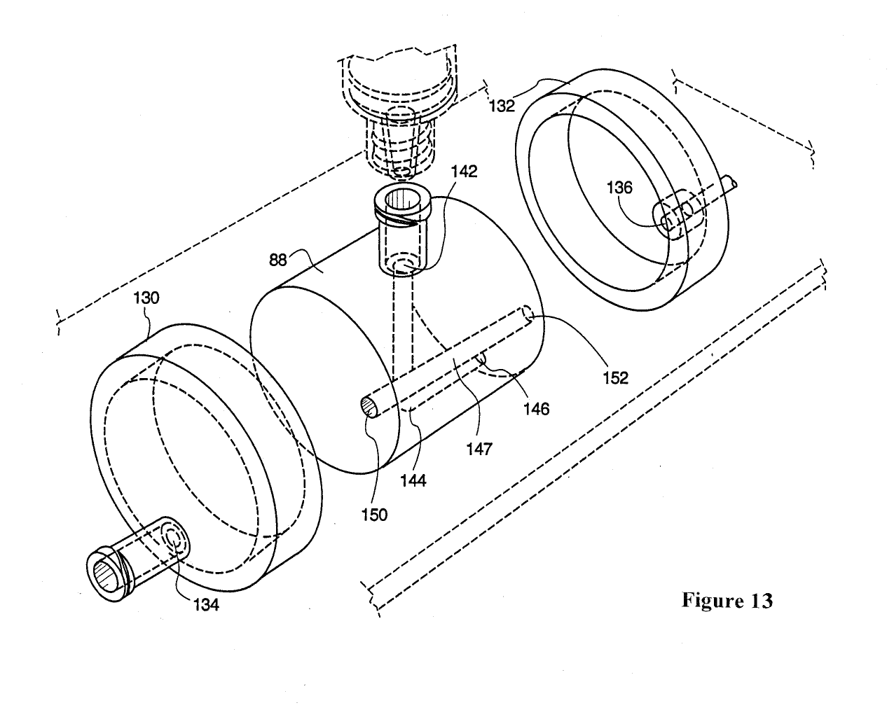

FIG. 13 shows an exploded perspective view of the injection valve in injection mode. In injection mode, the injection syringe is positioned perpendicular to the plane of base 93, as seen with valve 82b in FIGS. 1 and 2. This rotational position causes opening 146 in injection channel 144 to align with downstream opening 136 in downstream end cap 132. Injection mode allows fluid to be conducted from injection syringe 98 into injection cylinder port 142, through injection channel 144, out of cylinder opening 146, through end cap opening 136, then into the next injection valve, if one is present, and then into downstream conduit 104. Injection syringe 98 is not in fluid communication with the upstream fluid path in any mode, so there is no risk of upstream dilution or contamination of the contents of the injection syringe.

The embodiments described herein can improve the efficiency of routine and emergency administration of IV fluids. For example, a clinician can inject and then flush medicament to a patient using just one hand with a few fast steps and with enhanced protection against medication errors. Using certain stopcock technology, the number of two-handed steps to inject a medicament and then flush the line by syringe can be at least ten, with additional steps possibly required to clamp and unclamp auxiliary infusions, to fill a flush syringe from a separate reservoir, or to open a pre-filled flush syringe. The steps involving roller clamps can be particularly laborious and can add significant physical and mental labor to the process, given the frequency with which these tasks may be performed each day. The embodiments described herein can reduce the number of steps to seven, make them all one-handed, and eliminate any roller clamp manipulation. The steps can be, for example, beginning with all valves in drip mode: (1) injection syringe raised to vertical; (2) injection syringe plunger depressed to deliver medicament; (3) injection syringe lowered to horizontal; (4) flush syringe raised to vertical; (5) flush syringe plunger withdrawn to fill; (6) flush syringe plunger depressed to deliver flush; and (7) flush syringe lowered to horizontal.

As described herein, flush valve 80 physically separates two commonly required functions, drip flow and flushing, into mutually exclusive, for example, parallel, fluid channels—those being midstream conduit 94 and flush channel 120. Either channel can be placed into the fluid stream between upstream conduit 90 and downstream conduit 104 using one hand by moving flush syringe 96, which serves as a valve actuator. In other embodiments, a flush valve controls flow through multiple mutually exclusive fluid channels, not just two. Each fluid channel can be selected using a similar one-handed movement of a syringe actuator, aligning upstream and downstream openings of the selected channel with upstream and downstream end cap openings. Each channel, for example, configured in parallel with other channels, can serve a different clinical purpose by virtue of the modifications imposed on the fluid stream along its length. By way of example, one channel can be for flushing with saline, one for flushing with heparinized saline, one for routine infusions, one for emergency infusions, one for IV contrast solution, and so on.

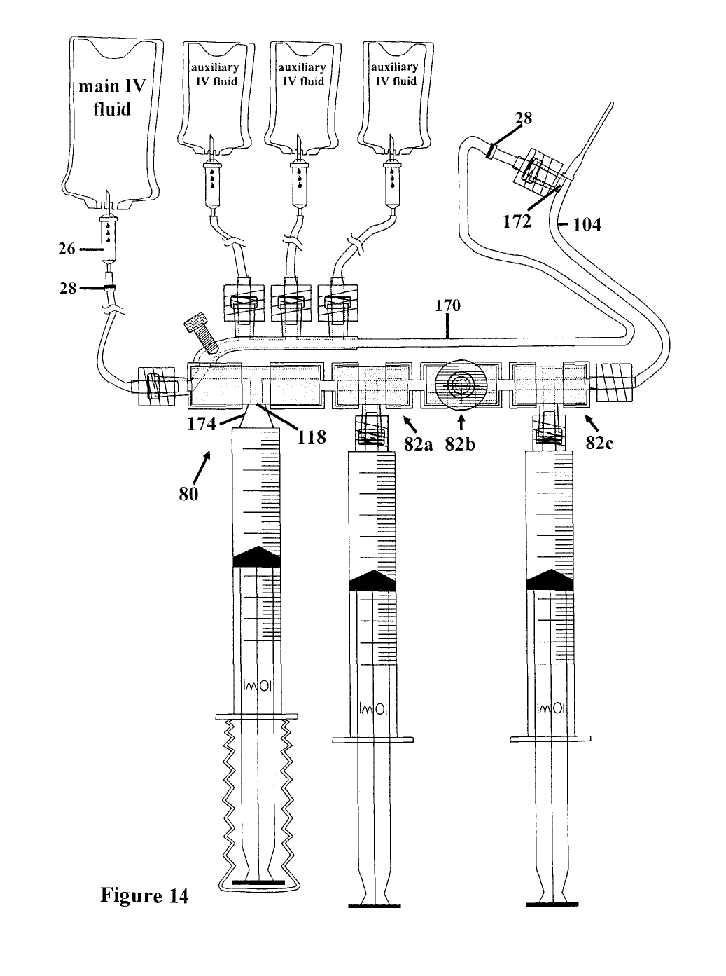

In other embodiments, shown in FIG. 14, the flush syringe is permanently fused to the flush cylinder at port 118, unable to be detached without damaging the syringe and/or the cylinder, by way of a rigid connector element 174. This feature would ensure that no clinician mistake the flush cylinder for an injection or infusion port, as no available free attachment port would exist on the cylinder.

FIG. 14 also shows the flush syringe plunger enclosed in an enclosure, such as a sealed deformable plastic casing 176, as shown. The casing, which can enclose the entire syringe or only a portion of the syringe (e.g., only the plunger), can include a flexible membrane that functions in a bellows-like manner. The casing can ensure a closed, sterile internal environment for the flush syringe, minimizing infection risk over multiple flushing tasks. Such an enclosed flushing system can ensure that the fluid in the flush syringe is as sterile as that in the upstream IV fluid reservoir.

FIG. 14 also shows an elongated midstream conduit 170 reentering the main fluid stream by joining with downstream conduit 104 near the patient's IV catheter at port 172 instead of reentering at the flush valve, as does midstream conduit 94 described above. A check valve 28 is shown allowing only unidirectional flow from midstream conduit 170 into downstream conduit 104. The portion of downstream conduit 104 which could contain auxiliary IV fluid is reduced, thereby reducing the amount of auxiliary IV fluid that could be delivered rapidly to the patient as a consequence of giving a fluid bolus through an injection valve or giving a flush through the flush valve. Reducing (e.g., minimizing) the potential for bolus administration of even small quantities of auxiliary IV fluid can be worthwhile, because some auxiliary infusion drugs can be harmful, particularly if given too quickly, such as antibiotics, vasoactive pressors, and potassium. The risk is greatest when the concentration of auxiliary fluid in downstream tubing 104 is high, as occurs when drip flow from the main IV fluid reservoir is set to a low rate. These features also serve to quickly reestablish the desired concentration of auxiliary IV infusions entering the patient by drip flow following an injection bolus or a flush maneuver. It is recognized that, as shown, every drug given by an injection syringe is to be actively flushed using the flush valve, and not flushed passively using drip flow. This is because any administration of medicament through injection valve 82 leaves undelivered a quantity of medicament residing in downstream conduit 104 but upstream of opening 172, which only can be delivered to the patient by actively flushing through the flush valve.

In some embodiments, the components (e.g., plastic components) of the flush valve and injection valves include structural elements to prevent the cylinders from being rotated to any more extreme position than required for drip-flow mode, flush-fill mode, or injection mode. This would facilitate switching modes by allowing the clinician simply to move the actuating syringe in an arc until it stopped. For example, if a valve cylinder has multiple parallel fluid channels, as described above, then multiple rotational positions may need to be set with ease. To achieve this, another structural element that can be provided is a détente at each or any given cylinder position. This tactile clue or indicator can indicate when the flush syringe is in the proper rotational position. Such structural elements can include a nub on the surface of the valve cylinder, which, when it encounters a small depression in the end cap during rotation, would serve to promote stopping of rotation, but which can be overcome easily by further force to rotate the syringe to other positions.

In some embodiments, in association with the valve systems described herein, a bracket is included that can hold base 93 tightly so as to immobilize the valve end caps for easier valve cylinder rotation. Such a bracket can be attached conveniently to an IV pole, desk, or hospital bed. Attachment using an adhesive backing on the base is another alternative. Yet in other embodiments, flush valve 80 and/or injection valve 82 are not affixed to any base, but rather supported only by their upstream and downstream attachments to the IV tubing.

The flush valves described herein can be used without using the injection valves in some embodiments. For example, the flush valves can be placed upstream of any type of injection port including, but not limited to, Y-sites and four-way stopcocks to achieve flushing of downstream tubing in a manner similar to that described above. Thus, one could administer a bolus of fluid to the patient not using the injection valve 82, but still retain the advantages of the flush valve 80. Also, the injection valve can be used without using the flush valve. For example, one or more injection valves can be used in an intravenous line to achieve bolus dosing of medicament in a manner similar to that described above but without the presence of the flush valve upstream.

The number of injection valves present downstream of the flush valve can be anywhere from none to many, depending on the needs of the user. In some settings, such as for a patient with uncomplicated needs, it may be desirable to have one flush valve and one injection valve present on the base. In other settings, such as for patients in the ICU or operating room, it may be desirable to have one flush valve and two or more injection valves. As shown above, three injection valves are presented to facilitate the description. In some embodiments, the function of injection valves 82 is achieved by other injection ports, such as, for example, four-way stopcocks and/or Y-sites. Flush valve 80 can function upstream of various types of injection ports, not only injection valve(s) 82.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. An apparatus for directing fluid flow to a subject, the apparatus comprising:
a first valve sized and dimensioned for directing the flow of intravenous fluid comprising:
an inlet capable of being in fluid communication with an intravenous fluid source;
an outlet capable of being in fluid communication with the inlet; and
a first port to be in fluid communication with a flush syringe;
wherein:
the first valve has an axis of rotation and is capable of being in a first mode in which the inlet is in fluid communication with the outlet through a first flow path, and in a second mode in which the inlet is in fluid communication with the outlet through a second flow path isolated from the first flow path; and
the first port is in fluid communication with the second flow path, and the flush syringe, when coupled to the first port, provides a first valve handle for selecting between the first mode and second mode by pivotal rotation of a longitudinal axis of the flush syringe in an arc about the axis of rotation of the first valve.

2. The apparatus of claim 1, wherein the first valve is associated with a base, and the flush syringe is capable of pivoting relative to the base.

3. The apparatus of claim 1, wherein the first valve further comprises a controller capable of controlling fluid flow through the first flow path.

4. The apparatus of claim 1, wherein the first flow path is capable of being in fluid communication with a second fluid source.

5. The apparatus of claim 1, wherein the first valve comprises a body comprising at least a portion of the first flow path and at least a portion of the second flow path, and rotation of the body allows the first valve to be in a selected mode.

6. The apparatus of claim 1, wherein the first valve comprises:
a body comprising:
a first channel;
a second channel; and
a third channel,
the first, second, and third channels capable of being isolated from each other;
wherein:
in the first mode, the first channel, the second channel, the inlet, the outlet, and the first flow path are in fluid communication; and
in the second mode, the third channel, the inlet, and the outlet are in fluid communication.

7. The apparatus of claim 1, wherein the first valve further comprises a tactile indicator capable of denoting a selected mode of the first valve.

8. The apparatus of claim 1 further comprising:
a second valve capable of being in fluid communication with the first valve, the second valve comprising:
a second inlet;
a second outlet capable of being in fluid communication with the second inlet; and
a port;
wherein the second valve is capable of being in a third mode in which the second inlet is capable of being in fluid communication with the second outlet, and in a fourth mode in which the second outlet is capable of being in fluid communication with the port.

9. The apparatus of claim 8, wherein the second valve is configured to engage with a fluid injector, and the injector and the port are capable of being in fluid communication.

10. The apparatus of claim 8, wherein the second valve is associated with a base, and the injector is capable of pivoting relative to the base.

11. The apparatus of claim 1 further comprising an intravenous catheter capable of being in fluid communication with the outlet.

12. An apparatus for directing intravenous fluid flow to a subject, the apparatus comprising:
a first valve sized and dimensioned for directing the flow of intravenous fluid comprising:
a first inlet capable of being in fluid communication with an intravenous fluid source;
a first outlet capable of being in fluid communication with the first inlet; and
a first port adapted to be in fluid communication with a flush syringe;
wherein the first valve has an axis of rotation and is capable of being in a first mode in which the first inlet is in fluid communication with the first outlet through a first flow path, and a second mode in which the first inlet is in fluid communication with the first outlet through a second flow path isolated from the first flow path, the first port is in fluid communication with the second flow path, and the flush syringe, when coupled to the first port, provides a first valve handle for selecting between the first mode and second mode by pivotal rotation of a longitudinal axis of the flush syringe in an arc about the axis of rotation of the first valve; and
a second valve sized and dimensioned for directing the flow of intravenous fluid capable of being in fluid communication with the first valve, the second valve comprising:
a second inlet;
a second outlet capable of being in fluid communication with the second inlet; and
a second port adapted to be in fluid communication with a medicament injection syringe;
wherein the second valve has an axis of rotation and is capable of being in a third mode in which the second inlet is capable of being in fluid communication with the second outlet, and a fourth mode in which the second outlet is capable of being in fluid communication with the second port, and the medicament injection syringe, when coupled to the second port, provides a second valve handle for selecting between the third mode and the fourth mode by pivotal rotation of a longitudinal axis of the medicament injection syringe in an arc about the axis of rotation of the second valve;
wherein the first flow path extends from the first inlet of the first valve to a position downstream of the second outlet of the second valve when the first valve is in the second mode.

13. The apparatus of claim 12, wherein the first valve further comprises a port in fluid communication with the second flow path.

14. The apparatus of claim 13, wherein the port is adapted to be in fluid communication with an injector.

15. The apparatus of claim 14, wherein the first valve is associated with a base, and the injector is capable of pivoting relative to the base.

16. The apparatus of claim 12, wherein the first valve further comprises a controller capable of controlling fluid flow through the first flow path.

17. The apparatus of claim 12, wherein the first flow path is capable of being in fluid communication with a second fluid source.

18. The apparatus of claim 12, wherein the first valve comprises a body comprising at least a portion of the first flow path and at least a portion of the second flow path, and rotation of the body allows the first valve to be in a selected mode.

19. The apparatus of claim 12, wherein the first valve further comprises a tactile indicator capable of denoting a selected mode of the first valve.

20. An intravenous fluid valve control system comprising:
a flush valve having an upstream end and a downstream end and having at least three actuating modes, such modes being:
a drip-flow mode, wherein the flush valve is in a first position and provides for constant flow in a first fluid path through the flush valve from an upstream fluid reservoir into the upstream end of the flush valve and out the downstream end of the flush valve;
a flush-fill mode, wherein the flush valve is in a second position different from the first position and provides flushing in a second fluid path through the flush valve from the upstream fluid reservoir into the flush syringe; and
a flush-purge mode, wherein the flush valve is in a third position different from both the first position and the second position and provides a third fluid path through the flush valve for allowing fluid in the flush syringe to pass out through the downstream end of the flush valve for purging.
